(19) United States
(12) Patent Application Publication
(10) Pub. No.: US 2010/0016822 A1
(43) Pub. Date: Jan. 21, 2010

(54) DISPOSABLE PERSONAL PRODUCT

(75) Inventor: Charishma Mohini Wickremesinghe, Cosmo (AU)

Correspondence Address: NIXON & VANDERHYE, PC, 901 NORTH GLEBE ROAD, 11TH FLOOR, ARLINGTON, VA, 22203, US

(73) Assignee: SENEVENS INTERNATIONAL PARTY LTD., Cosmo, WA (AU)

(21) Appl. No.: 12/302,106

(22) PCT Filed: May 23, 2006
(86) PCT No.: PCT/AU06/00675

§ 371 Date: May 11, 2009

(51) Int. Cl. A61F 13/53

(52) U.S. Cl. 604/375

(57) ABSTRACT

A disposable personal product such as a diaper comprising an absorbent core formed of starch grains dispersed in viscose fluff. The core may also comprise a small percentage of super absorbent polymer (SAP) to increase absorbency. The personal product further comprises an outer cover, a hydrophobic layer, a biodegradable film and a hydrophilic layer.

### FIELD OF THE INVENTION

The present invention relates to a disposable personal product and an absorbent composition for use therewith. The invention has been devised particularly, but not solely, for use in absorbing body waste discharges, and typically includes single use products such as infant diapers, feminine hygiene products, and incontinence products.

### BACKGROUND OF THE INVENTION

Articles used to absorb waste body fluids such as urine, are used in large quantities worldwide. Typically these articles are in the form of disposable diapers which have become increasingly popular as a modern convenience and necessity.

Large quantities of disposable infant diapers are used each year and disposal of these products is a problem. Most of the commercially available disposable diapers consist largely of polymer chemical products which do not readily break down in the environment.

A further difficulty associated with prior art disposable diapers is that due to the large quantities of chemicals in these products, rashes and skin allergies often arise in the user. Due to the requirements to maximise the absorbency of these products, the quantities of added chemicals, such as polyacrylate polymers, have been increased, further compounding the associated problems.

A number of prior art diapers are uncomfortable to wear as they are made of plastic material, and thus do not present a soft surface to the skin. Furthermore, these prior art diapers prevent natural air flow to the skin.

There is a need for a disposable personal product with an effective absorbency level, which is also environmentally friendly, and which does not typically cause an adverse reaction on contact with the skin of a user.

Thus it is an object of the present invention to overcome, or at least partially alleviate, the difficulties associated with the prior art.

The word “disposable” is used in the specification to describe absorbent articles which are not intended to be laundered or otherwise restored or reused as an absorbent article. They are intended to be discarded after a single use and preferably to be disposed of in an environmentally friendly manner.

The phrase “personal product” is intended to include products or articles used to absorb waste body fluids or other body waste, and includes diapers, nappies, absorbent underpants, training pants, adult incontinence products, feminine hygiene products and breast pads. This phrase is also intended to cover products used as protectors for beds, chairs and baby cots which may come into contact with waste body discharges.

References to prior art in this specification are provided for illustrative purposes only and are not to be taken as an admission that such prior art is part of the common general knowledge in Australia or elsewhere.

### SUMMARY OF THE INVENTION

The present invention provides a disposable personal product comprising an absorbent layer of viscose. Preferably the viscose is untreated viscose such as unbleached viscose. Preferably the viscose is in the form of viscose fluff. The viscose may be compressed viscose.

The absorbent layer preferably further comprises starch to form an absorbent core. The viscose may be coated with the starch to form the absorbent core. As a preferred alternative, the starch is in the form of starch grains dispersed within the viscose. The starch grains are preferably small, granular particles around the size of powdered sugar grains. The relative weight percentage of starch in the absorbent core is preferably about 7%.

The absorbent core may further comprise a small amount of a super absorbent polymer (SAP), polyacrylate or a polyacrylate salt, mixed with the starch to assist in absorption of the waste fluid matter. Preferably the SAP is present in the absorbent core in an amount of less than about 3% by weight of the absorbent core in the case of baby and infant diapers. Preferably the SAP is present in an amount of about 7 to 20% in the absorbent core in the case of adult diapers and incontinence products.

More preferably the absorbent core has a composition with relative weight percentages of viscose: starch: SAP of about 90%:7%:3%.

The absorbent core is preferably substantially rectangular in shape to conveniently collect urine and blood or other waste matter from the body of the user as excreted.

The starch used in the absorbent core is preferably formed by grafting a polymeric composition to corn starch.

The absorbent core may be surrounded by a layer of tissue paper.

The product may further comprise an outer layer in the form of an outer cover. The outer cover serves to form a covering for retaining the absorbent core and other components of the product. The outer cover is preferably formed of any one or more of cotton, viscose, silk or wool. The outer cover is preferably in the form of a non-woven felted fabric, such as a felted viscose fabric, and may be unbleached, bleached or partially bleached. The felted viscose fabric may be needle felted and may be in any one of the forms used in non-woven fabrics including random laid, parallel laid or cross laid. The use of the non-woven felted viscose fabric provides a soft, smooth feel to the skin.

The product may further comprise a hydrophobic layer adjacent skin of the user to assist in repelling liquid away from the skin of the user. The product may optionally include a hydrophilic layer beneath the hydrophobic layer to assist in preventing the waste liquid absorbed by the absorbent core from being drawn back towards the skin. The product may also comprise a biodegradable sheet film to assist in waterproofing and retaining liquid matter within the product. The biodegradable film is typically positioned adjacent the absorbent core to assist in preventing the moisture from seeping through the outer cover.

In a further preferred form of the invention, the invention provides a disposable personal product comprising an absorbent core, the absorbent core comprising viscose and starch. The starch is preferably in the form of starch grains, the grains being dispersed within the viscose. The starch grains are preferably present in an amount of about 7% by relative weight of the absorbent core. The absorbent core may further comprise a small percentage of SAP also dispersed within the viscose.

Still further, the invention provides a disposable personal product comprising an outer cover, a hydrophobic layer adjacent skin of a user, and an absorbent core intermediate the outer cover and the hydrophobic layer, the absorbent core comprising viscose and starch. The starch is preferably in the form of starch grains dispersed in the viscose. The product may further comprise an inner hydrophilic layer positioned beneath the hydrophobic layer.

The invention further provides a composition for a disposable personal product comprising an absorbent layer of unbleached viscose. The absorbent layer may further comprise starch to form an absorbent core. The starch is preferably in the form of grains disposed in the viscose.

Throughout the specification, unless the context requires otherwise, the word “comprise” or variations such as “comprises” or “comprising”, will be understood to imply the inclusion of a stated integer or group of integers but not the exclusion of any other integer or group of integers. Likewise the word “preferably” or variations such as “preferred”, will be understood to imply that a stated integer or group of integers is desirable but not essential to the working of the invention.

### BRIEF DESCRIPTION OF THE DRAWINGS

The present invention will now be described, by way of example only, with reference to the accompanying drawings, in which:

FIG. 1 is a schematic plan view of a diaper according to an embodiment of the invention;

FIG. 2 is a schematic cross-sectional view through the diaper of FIG. 1;

FIG. 3 is a perspective view of the diaper of FIG. 1 in the laid flat condition viewed from the exterior;

FIG. 4 is a perspective view of the diaper of FIG. 1 in the laid flat condition as viewed from the interior; and

FIG. 5 is a perspective view of the diaper of FIGS. 3 and 4 in the as-worn condition.

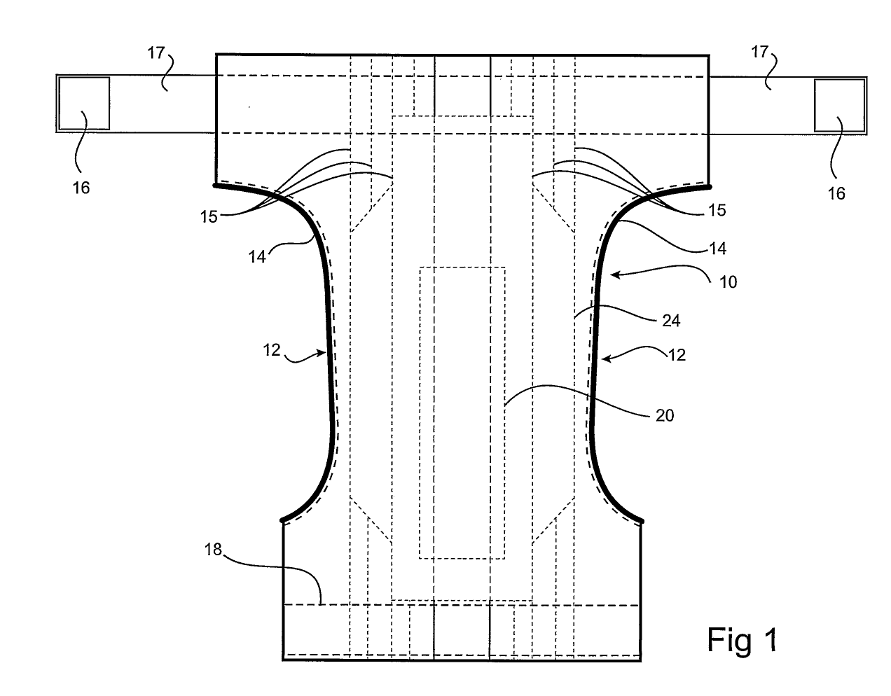

### DESCRIPTION OF AN EMBODIMENT OF THE PRESENT INVENTION

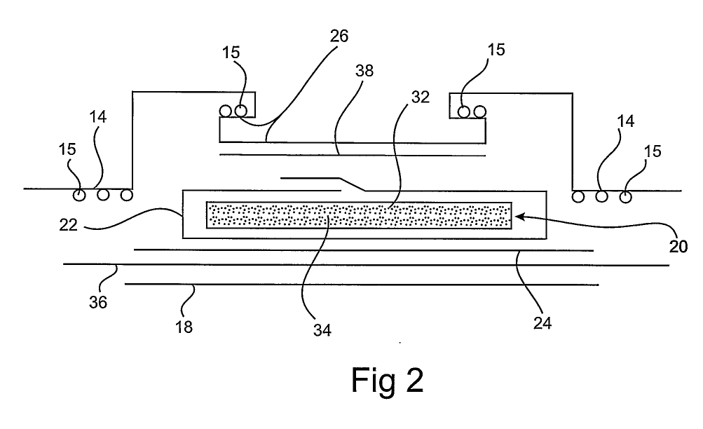

A diaper 10 for an infant having a pair of shaped leg portions 12 and elasticised cuff portions 14 is shown in the Figures. As can best be seen in FIG. 2, the cuff portions 14 have three rows of elastic 15 running the length of the diaper 10 so that the edges of the cuff portions 14 sit securely on the legs of the infant. Two further rows of elastic 15 may be provided on an extension of the cuff section 14 of the diaper 10 to assist in holding the diaper in position on the legs as best seen in FIG. 2.

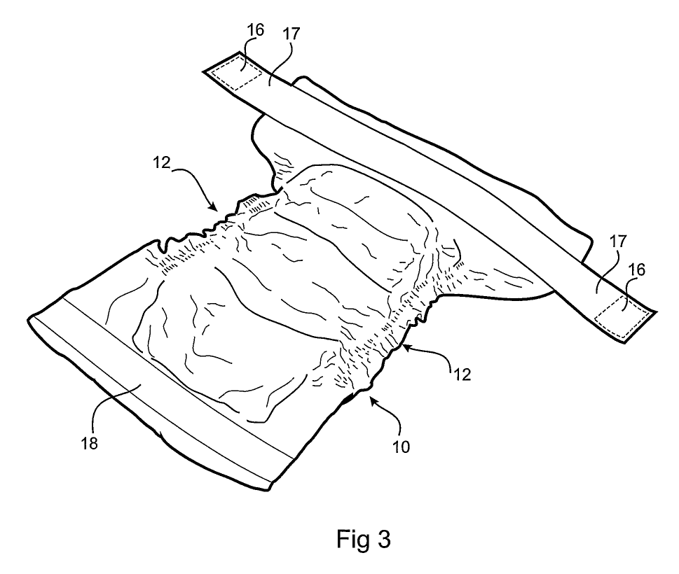

The diaper has a banded portion 17 extending beyond the upper edge of the exterior of the diaper as shown in FIG. 3. The banded portion 17 is provided with adhesive tab portions 16 for fixing to the tape portion 18 to secure the diaper during use.

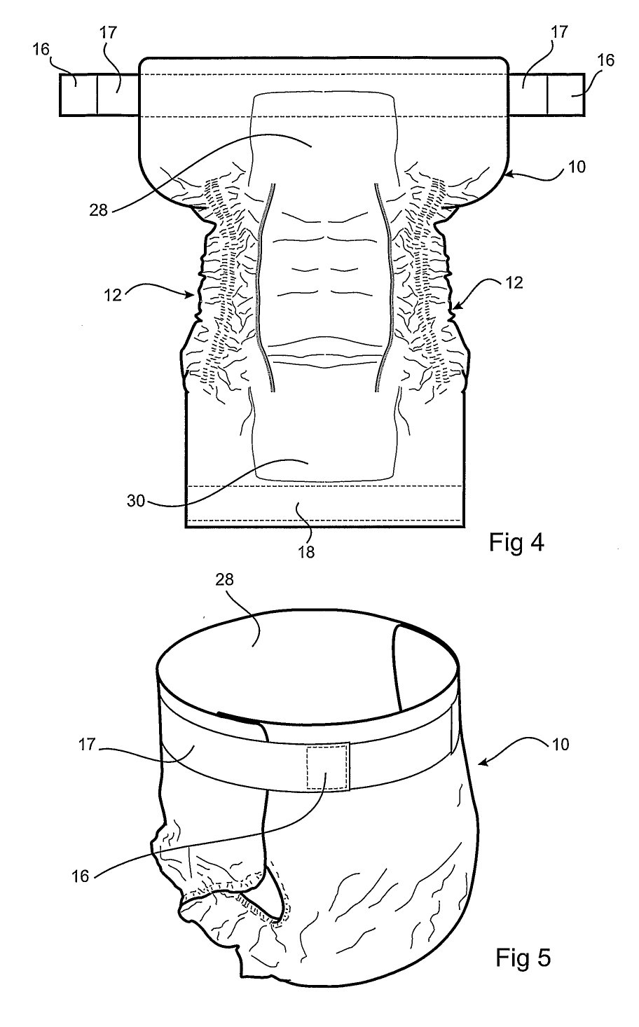

In use, an infant (not shown) is placed on the diaper 10 in the laid flat condition as shown in FIG. 4, with its legs positioned in the leg portions 12 and its back resting on the inner upper portion 28 of the diaper 10. The inner lower portion 30 of the diaper 10 is folded upwards towards the abdomen of the infant and the banded portion 17 is drawn around the front of the diaper so that the first tabs 16 on the banded portion 17 match up with the tape portion 18. To secure the diaper, the first tabs 16 are provided with adhesive (not shown) to secure the tape portion 18 allowing the diaper 10 to be held in a secure manner in use as shown in FIG. 5.

The diaper 10 has an absorbent core 20 comprised of starch grains 32 dispersed in viscose fluff 34 as shown in FIG. 2. The starch grains 32 are small granular particles of similar size to typical sugar grains. The absorbent core also includes superabsorbent polymer (SAP) (not shown in the drawings). The relative weight percentage of viscose: starch: SAP in the absorbent core 20 is about 90%:7%:3%. The presence of the SAP assists in increasing the absorbency of the product but since it is present in such a relatively small amount the usual adverse effects of the SAP found in prior art diapers are not significant. The usual level of SAP in prior art diapers is about 40%. Large amounts of SAP have caused the health and environmental problems previously discussed. In the case of products of the present invention the content of SAP is usually only around 3%. Thus, the quantity of SAP used in the present invention is at a sufficient level to assist in absorption of fluid but is not at a high enough level to cause dehydration of body moisture.

In the case of diapers used for babies of infants, the typical percentage of SAP present is less than or equal to about 3%. In the case of adult diapers, incontinence pads, or sanitary products, the absorbent core preferably comprises around 7 to 20% of SAP due to the larger volume of liquid which needs to be absorbed.

The presence of starch grains dispersed in the viscose fluff provides an effective means for the absorption of fluids in the product. It is thought that when the water or waste fluid falls on the starch grains, polar repulsion between the molecules occurs which results in swelling of the particles and absorption of the water or fluid.

Starch in the form of grains provides an increased surface area for contact with moisture and assists in increasing the absorbency capacity of the diaper. The effect of incorporating the starch grains 32 in the viscose fluff 34 is to create a material that acts like a sponge capable of high absorption rates. The starch is capable of sustaining multiple “swell-shrink” cycles.

Viscose is a regenerated fibre. Cellulose fibre in its regenerated form is used to produce viscose. Viscose usually contains about 10 per cent moisture when “dry” and may absorb up to about 21 per cent moisture before feeling wet. Thus it provides a convenient substance for use in diapers and other like products that have a need for the absorption of fluids.

The main absorbent ingredient in the absorbent core is the starch in the form of starch grains. The starch used in the absorbent core is preferably formed by grafting a polymeric composition to corn starch molecules. Due to the nature of the starch, it is capable of absorbing up to 300 times its weight in water. The additional starch is the form of grains is substantially bio degradable and compostable.

The absorbent core 20 is surrounded by a layer of tissue paper 22.

The diaper 10 is provided with an outer cover 36 formed of non-woven felted viscose although other materials may also be suitable. The outer cover is soft and has a comfortable feel against the skin of a user. The nature of this fabric allows for breathability and ease of air flow around the skin of a user. The feel of the outer cover 36 is very smooth to the body of the user and provides ease of movement and natural air flow to the body. The natural fibre construction of the outer cover offers maximum health and environmental benefits.

The diaper 10 also has a hydrophobic layer 26 of non-woven material and an inner hydrophilic layer 38. The hydrophobic layer 26 provides for a substantially dry area next to the skin of the user. The hydrophobic layer 26 is typically a bonded, non-woven fabric. The elastic 15 is also of a hydrophobic nature and may be formed of the same bonded, non-woven fabric, which further assists in retaining dryness next to the skin.

The diaper 10 is also conveniently provided with a biodegradable sheet film 24 which assists in waterproofing and retaining the absorbed fluid within the diaper 10. The biodegradable film 24 is typically positioned adjacent the absorbent core 20 as can be seen in FIG. 2, to assist in preventing the moisture from seeping through the outer cover. The film 24 is a product based on a vegetable oil and is a breathable and waterproof film layer and therefore is beneficial to the overall design of the diaper 10. Alternatively, the film 24 may be in the form of a wax such as a natural wax or water based wax.

Now that preferred embodiments of the disposable personal product have been described in detail, it will be apparent that it provides a number of advantages over the prior art, including the following:

(i) It provides an effective personal product comprised of substantially natural or regenerated/reconstructed materials or fibres such that on disposal there is minimum adverse impact on the environment;

(ii) The personal product is formed of materials and fibres to provide an effective and healthy product for the user;

(iii) The invention provides an environmentally friendly and generally allergy free composition and product due to the use of natural and regenerated materials;

(iv) The soft outer cover of non-woven viscose presents a feeling of softness to the skin. The fabric allows ease of air circulation and breathability around the skin of the user; and

(v) The use of the biodegradable film layer assists in retaining fluid in the diaper whilst at the same time conforming to the requirements of a natural environmentally friendly product.

It will be readily apparent to persons skilled in the relevant arts that various modifications and improvements may be made to the foregoing embodiments, in addition to those already described, without departing from the basic inventive concepts of the present invention. For example, the outer cover of the personal product may be comprised of materials other than viscose that also give a soft feel to the product, such as cotton, rayon, silk or wool, or mixtures thereof.

Therefore, it will be appreciated that the scope of the invention is not limited to the specific embodiments described.

1. A disposable personal product comprising an absorbent layer of viscose.

2. A disposable personal product according to claim 1 wherein the viscose is unbleached viscose.

3. A disposable personal product according to claim 1 wherein the viscose is a viscose fluff.

4. A disposable personal product according to claim 1 wherein the viscose is compressed viscose.

5. A disposable personal product according to claim 1 wherein the absorbent layer further comprises an absorbent core including starch.

6. A disposable personal product according to claim 5 wherein the viscose is coated with the starch to form the absorbent core.

7. A disposable personal product according to claim 5 wherein the starch is in the form of starch grains.

8. A disposable personal product according to claim 7 wherein the starch grains are dispersed within the viscose.

9. A disposable personal product according to claim 5 wherein the relative weight percentage of viscose:starch in the absorbent core is about 90%:7%.

10. A disposable personal product according to claim 5 wherein the absorbent core further comprises at least one of a super absorbent polymer (SAP), polyacrylate and polyacrylate salt, mixed with the starch.

11. A disposable personal product according to claim 10 wherein the SAP in the absorbent core is present in an amount of less than about 3%.

12. A disposable personal product according to claim 10 wherein the SAP is present in the absorbent core an amount of about 7 to 20%.

13. A disposable personal product according to claim 10 wherein the absorbent core has a composition with relative weight percentages of viscose: starch: SAP of about 90%:7%:3%.

14. A disposable personal product according to claim 5 wherein the absorbent core is substantially rectangular in shape.

15. A disposable personal product according to claim 5 wherein the starch grains are small, granular particles around the size of powdered sugar grains.

16. A disposable personal product according to claim 5 wherein the starch is formed by grafting a polymeric composition to corn starch.

17. A disposable personal product according to claim 5 wherein the absorbent core is surrounded by a layer of tissue paper.

18. A disposable personal product according to claim 1, the product further comprising an outer layer in the form of an outer cover, the outer cover comprising at least one of cotton, viscose, silk and wool.

19. A disposable personal product according to claim 18 wherein the viscose is a non-woven felted fabric.

20. A disposable personal product according to claims 19 wherein the felted fabric is a needle felted fabric.

21. A disposable personal product according to claim 1, wherein the product further comprises a hydrophobic layer adjacent skin of the user.

22. A disposable personal product according to claim 21, wherein the product further comprises a hydrophilic layer beneath the hydrophobic layer.

23. A disposable personal product according to claim 5 wherein the product further comprises a biodegradable sheet film positioned adjacent the absorbent core.

24. A disposable personal product comprising an absorbent core, the absorbent core comprising viscose and starch.

25. A disposable personal product according to claim 24 wherein the starch is in the form of starch grains, the grains being dispersed within the viscose.

26. A disposable absorbent product according to claim 25 wherein the starch grains are present in an amount of about 7% by relative weight of the absorbent core.

27. A disposable personal product comprising
an outer cover,
a hydrophobic layer adjacent skin of a user, and
an absorbent core intermediate the outer cover and the hydrophobic layer, the absorbent core comprising viscose and starch.

28. A disposable personal product according to claim 27 wherein the starch is in the form of starch grains dispersed in the viscose.

29. A disposable personal product according to claim 27 wherein the absorbent core further comprises SAP.

30. A disposable personal product according to claim 27 wherein the product further comprises a hydrophlic layer positioned beneath the hydrophobic layer.

31. A composition for a disposable personal product comprising an absorbent layer of unbleached viscose.

32. A composition according to claim 31 wherein the absorbent layer further comprises starch to form an absorbent core.

33. A composition according to claim 32 wherein the starch is in the form of grains disposed in the viscose.

34.-35. (canceled)
